(19) United States
(12) Patent Application Publication
(10) Pub. No.: US 2004/0260327 A1
(43) Pub. Date: Dec. 23, 2004

(54) Adjustable dilator assembly

(76) Inventors: Richard L. Mueller JR., Jackson, WY (US); Mallory Factor, New York, NY (US)

Correspondence Address: OLSON & HIERL, LTD., 36th Floor, 20 North Wacker Drive, Chicago, IL, 60606, US

(21) Appl. No.: 10/602,173

(22) Filed: Jun. 23, 2003

(51) Int. Cl. A61M 29/00

(52) U.S. Cl. 606/191

(57) ABSTRACT

An adjustable dilator assembly suitable for the dilation of a mammary duct includes an elongated hollow housing that terminates at one end into a cannula and defines a through passageway. A pliant probe, extendable though the catheter, is mounted within the hollow housing for adjustable axial movement. The probe is preferably mounted to a probe support or carrier that is threadedly engaged with the hollow housing.

### TECHNICAL FIELD OF THE INVENTION

The invention relates to a device for dilating a mammary duct. In particular, the invention relates to an adjustable dilator assembly suitable for the dilation of a mammary duct that includes an elongated hollow housing which terminates at one end into a cannula and defines a through passageway.

### BACKGROUND OF THE INVENTION

Breast cancer is one of the health threats most feared by women, and is a common form of cancer in women. In rare instances the human male may also have occurrences of breast cancer. A key to treatment of breast cancer is early detection. For example, an annual mammogram is a method that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. As such, more sensitive and reliable methods and devices are needed to detect cancerous, pre-cancerous, and other cancer markers of the breast at an early stage. Such methods and devices could significantly improve breast cancer survival.

Some methods of detecting breast cancer are based on the fact that a vast majority of instances of breast cancer begins in the lining of mammary ducts. Studies have shown that fluid within the mammary duct contains high levels of breast cancer markers, and that an estimated 80%-90% of all breast cancers occur within the intraductal epithelium of the mammary glands. Fluid within the breast ducts contains an assemblage and concentration of hormones, growth factors and other potential markers comparable to those secreted by, or acting upon, the surrounding cells of the alveolar-ductal system. Likewise, mammary duct fluid typically contains cells and cellular debris, or products that can also be used in cytological or immunological assays. As such, various method for collection of mammary duct fluid and cellular debris therein have been developed.

One such method of obtaining samples of cells from a mammary duct is ductal lavage. This method comprises the introduction of a rinsing solution, such as a saline solution or the like, into a mammary duct, and the collection of the solution along with any cells and cellular debris from the mammary duct. Typically, a catheter or cannula having an internal lumen is used to introduce the solution into the mammary duct. Conventional catheters and cannulas include distal portions that may be introduced into a mammary duct via a nipple orifice terminating at the surface of a human nipple, and advanced with the assistance of an internally positioned dilator. The use of a dilator is preferred inasmuch as catheters and cannulas have open ends with edges that can harm a patient. This is because mammary ducts terminate at the surface of a human nipple at an orifice and are normally closed by a sphincter muscle which must be bypassed. A catheter's or cannula's open end, however, usually terminates at sharp angles or narrow edges that can damage a mammary duct as well as the sphincter muscle. A dilator, which may pass through the lumen of a catheter or cannula, does not have an open end.

Conventional dilators are made of a substantially rigid metal or hard plastic. In use, the dilator is extended through the distal end of a catheter or cannula and past the ductal sphincter muscle. Alternatively, a dilator is inserted into a mammary duct and the catheter or cannula is then guided over the dilator. The dilated sphincter is then more easily passed by the catheter or cannula. Also, since the dilator is sized only slightly smaller than the lumen, the sharp distal end of the catheter or cannula is less likely to damage the mammary duct or the sphincter muscle. The dilator can then be removed from the lumen in order to flush the mammary duct with a lavage solution or to withdraw existing mammary duct liquid.

Mammary duct damage, however, may still occur when rigid dilators are utilized due to their rigid nature. Use of softer pliable materials to dilate mammary ducts, such as a polypropylene monofilament guiding suture, has been tried with limited success.

An improved atraumatic dilator is disclosed in co-pending U.S. application Ser. No. 10/350,631. The atraumatic dilator comprises a filament having a proximal end portion and a distal end portion. At least one end portion of the filament is of a pliable material. The end portions of the atraumatic dilator can be tapered and also can have a rounded tip. In certain situations, however, where a polymer is too soft or pliable, it will not have the lateral strength to resist merely being bent as one attempts to insert the dilator past the sphincter muscle. Accordingly, various stiffeners that may be used in conjunction with the atraumatic dilator are also disclosed in co-pending U.S. application Ser. No. 10/350,631.

A need still exists, however, for an adjustable dilator, wherein the probe for dilating the mammary duct is such that it is not likely to traumatize the mammary duct, nipple orifice or sphincter muscle, yet can provide sufficient rigidity to penetrate and pass the sphincter muscle. The present device meets these needs.

### SUMMARY OF THE INVENTION

An adjustable dilator assembly suitable for use in mammary ducts is provided. The adjustable dilator assembly comprises a hollow housing that terminates at one end into a cannula, a probe sized to be received within the cannula, and preferably a probe carriage movably situated within the housing. The probe carriage and the probe associated therewith are adjustably positionable axially with the housing and the cannula, respectively.

The hollow housing includes a handle portion having a through passage and a cannula portion which is an elongated hollow sheath mounted to and extending distally away from the handle portion. The sheath terminates at an open distal end. The elongated sheath may be secured within the through passage about the distal end portion of the handle portion. Alternatively, the elongated sheath and the handle portion can be integral or unitary. The handle portion is preferably made of polypropylene, polyethylene, polyurethane, or a like material of construction. The sheath is made of a relatively rigid material such as stainless steel or the like. If desired, the sheath and handle portion can be made of the same material.

The probe is relatively flexible and has a distal end portion that is extendable through the sheath and past the open distal end of the sheath. The inside diameter of the sheath is such that the probe can be readily inserted into and removed therefrom. Preferably, at least the distal end portion of the probe is pliable. The distal end portion further preferably includes a taper and terminates in a rounded tip. In some embodiments, the distal end portion may be heat bent so as to be offset laterally when in a relaxed state. A proximal end portion of the probe is removably or fixedly secured to a probe carriage, which in turn is adjustably engageable with the handle portion of the hollow housing. In embodiments where the probe and the probe carriage are attached to one another, the attachment can be integral or unitary. For example, both the probe and the probe carriage can also be made of polypropylene, polyethylene, polyurethane, or the like material.

The probe carriage is axially movable within the handle portion. Preferably, the handle portion and the probe carriage are suitable for threadable engagement with one another. In one embodiment, the handle portion defines threads along a portion of its interior, and the probe carriage has external threads along a portion of its length suitable for threaded engagement therewith. The internal threads of the handle portion and the external probe carriage threads are engageable with one another such that the probe carriage may be axially advanced within the hollow housing and the probe within and through the cannula portion. As the probe carriage is advanced within the handle portion, the probe carriage is increasingly extended from the sheath of the cannula portion. As an alternative to threaded engagement, the housing portion and the probe carriage can be in a telescoping relationship, frictionally engaged with one another. For example, the probe carriage and the handle portion can form two mating telescoping parts. Any suitable means of adjustably positioning the handle portion and cannula portion relative to the probe carriage can be used.

Typically, the sphincter muscle of a mammary duct may be bypassed with a pliable probe. However, in some situations, resistance to the probe causes the pliable probe to deflect and not penetrate the sphincter muscle. In such instances, to penetrate and dilate the nipple orifice as well as the sphincter muscle, the distal end portion of the probe is only partially extended, such that preferably about ⅛ of an inch (about 5.6 mm) extends beyond the distal end of the cannula portion. Because the pliable distal end portion of the probe is extended as such, the minimally exposed end portion of the pliant probe is nevertheless relatively stiff, or substantially rigid, and deflection of the distal end portion as it is used to penetrate the sphincter muscle of a mammary duct orifice is significantly reduced or eliminated.

Before penetrating the nipple orifice, it is first located by any convenient means, such as an illuminated nipple cup, a magnifier, or the like. The located nipple orifice is then penetrated with the present adjustable dilator assembly by guiding the exposed distal end portion of the probe past the sphincter muscle. The probe can then be extended further by adjusting the position of the probe carriage within the handle portion such that the distal end portion of the probe is extended further into a mammary duct. The probe is pliable, and as the probe is extended beyond the distal end of the sheath, the exposed length of the probe may be passed along the mammary duct without damaging the duct. The cannula may also be further advanced into the mammary duct along the path of the probe.

After the cannula portion is positioned within the mammary duct as desired, the probe may be removed from the mammary duct by disengaging the probe carriage from the handle portion and withdrawing the probe carried therewith from the cannula. An injection or ductal lavage device can then be brought into operative fluid communication with the positioned cannula portion. For example, in a preferred embodiment, the through passage of the handle portion is provided at the proximal end thereof with a female luer taper suitable for accommodating a standard syringe. The syringe can introduce a flushing or rinsing solution, such as saline, an anaesthetic such as lidocaine, or the like, into the mammary duct. The same syringe can then withdraw the rinsing solution and any particulate matter, which may include cytological samples, from the duct. The injection device is also suitable for extracting samples of existing liquid from the mammary duct without previously introducing a rinsing solution. Alternatively, an injection device can be provided integral with the probe carriage. For example, a bulb or balloon can be integral with the probe carriage and in fluid communication with the catheter portion for introducing or aspirating fluid from a mammary duct.

As yet another alternative, the probe may be removable from the probe carriage while the probe carriage is engaged with the housing. In such an embodiment, it is not necessary to remove the probe carriage from the housing to introduce liquid to or to remove liquid from the mammary duct with the injection device. For example, the probe carriage may be formed with a socket having a female luer taper, such that the injection mechanism, such as a syringe, may be connected directly to the probe carriage.

Depending upon configuration and surface characteristics, the probe may serve several functions, e.g., not only as an adjustable dilator but also as a device for collecting cytology samples from within a mammary duct.

### BRIEF DESCRIPTION OF THE DRAWINGS

In the drawings,

FIG. 1 is a side view of a preferred embodiment of an adjustable dilator assembly with portions in section to show interior detail;

FIG. 2 is an enlarged cross sectional view of the hollow housing of the dilator assembly of FIG. 1;

FIG. 3 is a side view of a probe and probe carriage of the dilator assembly of FIG. 1;

FIG. 4 is an enlarged schematic view of the cannula portion of an adjustable dilator assembly before insertion into a mammary duct with breast tissue shown in cross section;

FIGS. 5A and 5B are side schematic views showing a portion of the adjustable dilator assembly of FIG. 1 as it penetrates a human breast, shown in cross section, with the probe partly extended in FIG. 5A and fully extended in FIG. 5B;

FIG. 6 is a side view, partially in section, of the hollow housing of an adjustable dilator assembly embodying the present invention and mated with a syringe as an injection device;

FIG. 7 is a side view, partly in section, of an alternate embodiment of an adjustable dilator assembly having an injection device engaged therewith;

FIG. 8 is a side view, partly in section, of an alternate embodiment of an adjustable dilator assembly having an alternative injection device engaged therewith;

FIG. 9 is an enlarged partial side view of the distal end portion of the adjustable dilator assembly, partly in section, with an alternative probe;

FIG. 9A is an enlarged partial side view of the distal end portion of the adjustable dilator assembly, partly in section, with the probe of FIG. 9 retracted within the sheath;

FIG. 10 is a side view, partly in section, of an alternate embodiment of an adjustable dilator assembly having a probe integral with an injection device;

FIG. 11 is an enlarged partial side view of the distal end portion of a probe; and

FIG. 12 is a cross sectional view of the distal end portion of the probe of FIG. 11 taken at plane 12-12 in FIG. 11.

### DESCRIPTION OF PREFERRED EMBODIMENTS OF THE INVENTION

The invention disclosed herein is, of course, susceptible of being embodied in many different devices. Shown in the drawings and described herein below in detail are preferred embodiments of the invention. It is to be understood, however, that the present disclosure is an exemplification of the principles of the invention and does not limit the invention to the illustrated embodiments.

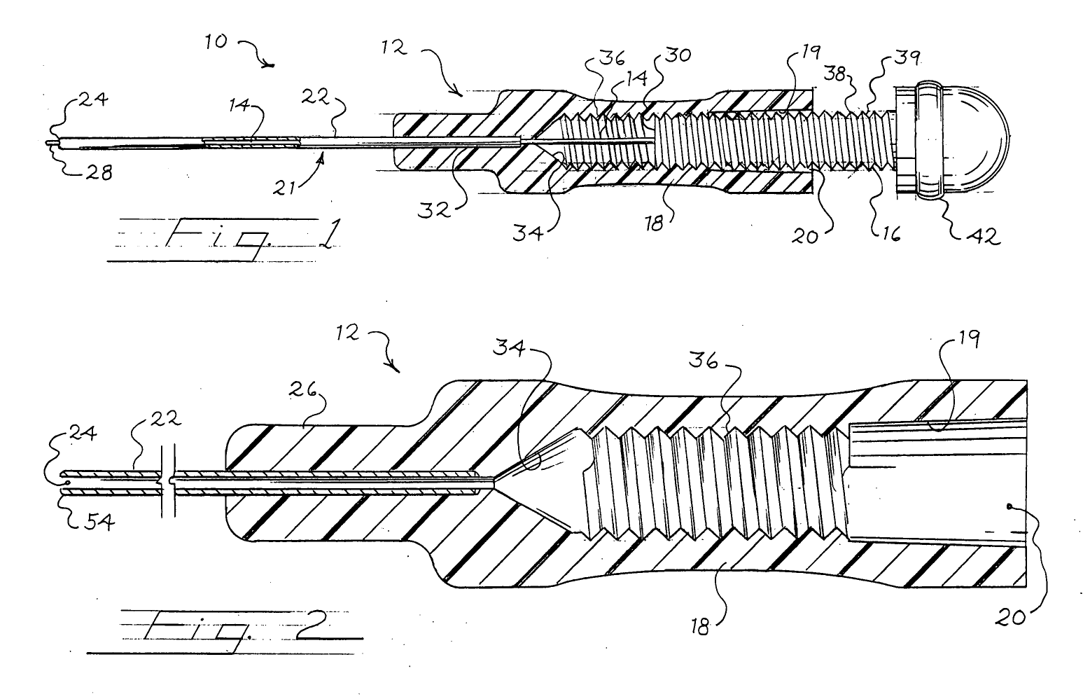

A preferred embodiment of the adjustable dilator assembly 10 is shown in FIG. 1. The adjustable dilator assembly 10 comprises an elongated hollow housing 12, a probe 14 and a probe carriage 16 carrying the probe 14. Hollow housing 12 terminates at one end in cannula 21 which is sized to receive probe 14 therewithin, defines an axial through passageway and has an opposite open end for receiving probe carriage 16.

Hollow housing 12 includes a handle portion 18 having an axial through passage 20. Cannula 21 comprises elongated hollow sheath 22 that extends distally from the handle portion 18. The sheath 22 is mounted to handle portion 18 and terminates at an open distal end 24. The elongated sheath 22 may be secured within the axial through passage 20 at the distal end portion 26 of the handle portion 18, as shown. Alternatively, the elongated sheath and the handle portion can be unitary with one another.

The probe 14 is received within cannula 21 and has a distal end portion 28 that is extendable along the through passage 20 and past the open distal end 24 of the sheath 22. Preferably, at least the distal end portion 28 of the probe 14 is pliable and has a rounded, atraumatic tip. In the embodiment shown, the probe 14 is pliable over its entire length, and has an atraumatic rounded tip provided with a taper. A proximal end portion 30 of the probe 14 is fixedly secured to probe carriage 16. Other probe variants are possible and are discussed hereinbelow.

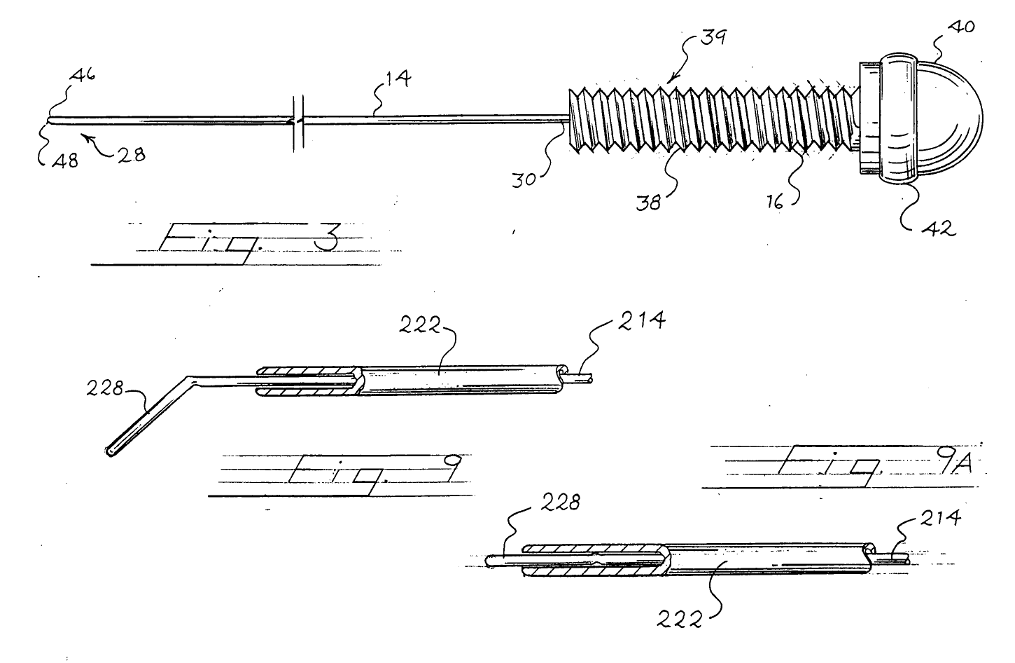

A preferred embodiment of housing 12 is described in greater detail with reference to FIG. 2. The proximal end portion 32 of sheath 22 is secured within through passage 20 about the distal end portion 26 of the handle portion 18. Through passage 20 includes a tapered portion 34 and an internally threaded portion 36 for threadably receiving external threads 38 on distal end portion 39 of probe carriage 16 (FIG. 3). A female luer taper 19 is also defined in through passage 20 at proximal end portion of the handle portion 18.

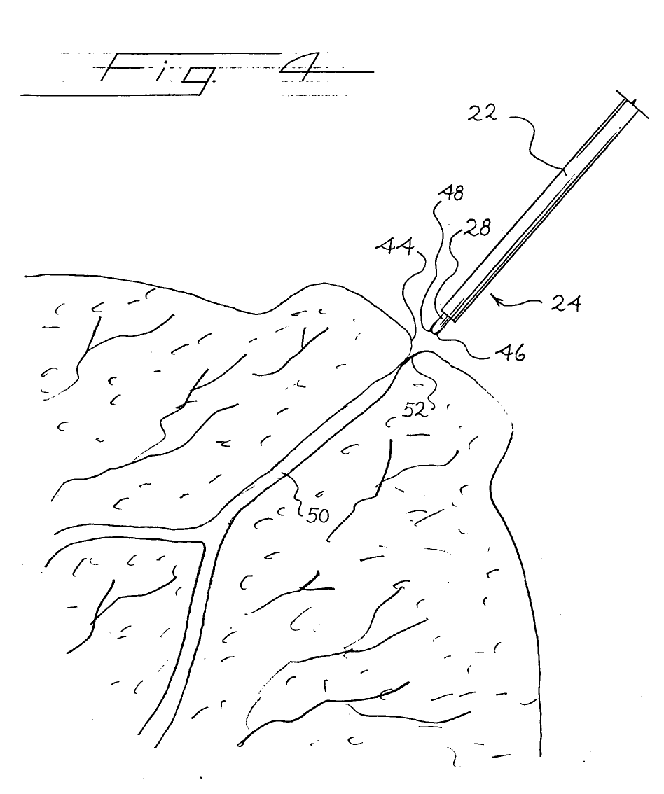

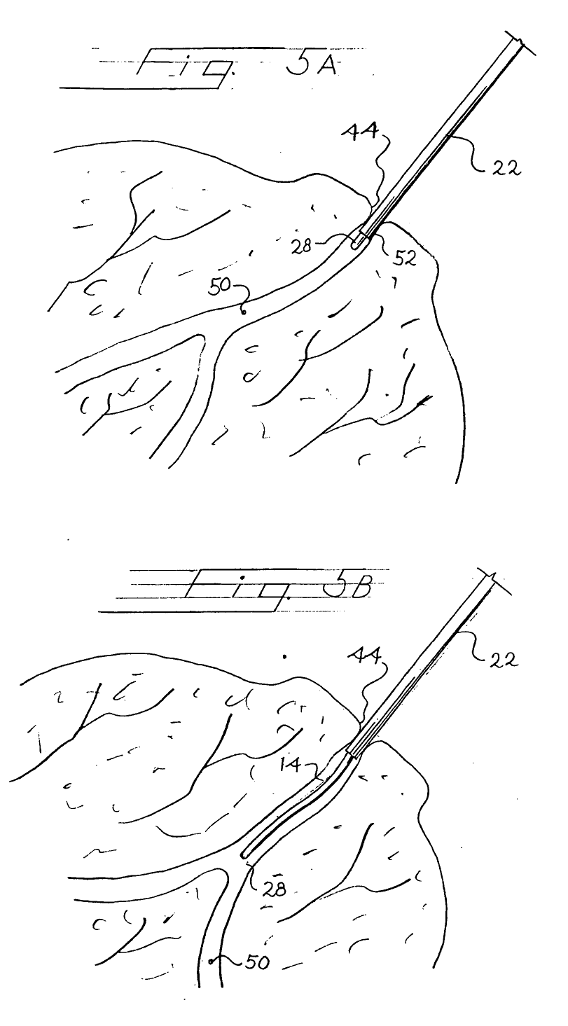

Referring to FIG. 3, probe 14 can be fixedly or removably secured to probe carriage 16 as desired. Probe carriage 16 also includes a grip such as knob 40 at the proximal end with which a practitioner manipulates by rotation the probe carriage relative to the handle portion 18 (FIG. 2) and thereby causes axial movement of probe 14 so as to be extended or withdrawn into cannula 21(FIG. 2). The distal end portion 28 of probe 14 can be extended beyond the distal end 24 of the sheath 22 sufficiently to serve as a cytology sample collector, as is discussed in further detail below. Preferably, knob 40 includes an annular grip 42 for enhanced control. Annular grip 42 is made of a rubberized material or the like. The distal end portion 28 of probe 14 also preferably includes a tapered end portion 46 and a rounded tip 48 (FIG. 3). The rounded tip 48 may be made of the same material as the distal end portion 28 of the probe 14, or may be made of a different material. The rounded tip 48 may be made of a relatively softer material to further lessen the possibility of duct injury during use. Preferably, the distal end portion 28 of the probe 14 includes a tapered portion 46 with a taper having an angle with the longitudinal center line of the probe (draft) in the range of about 2 degrees to about 15 degrees, and most preferably in the range of about 2 degrees to about 4 degrees. That is, the included angle of the taper more preferably is about 4 to about 30 degrees, most preferably about 4 to about 8 degrees. The tapered portion 46 and a rounded tip 48 of distal end portion 28 of probe 14 reduce the likelihood of trauma to the nipple orifice 44, mammary duct 50, or sphincter muscle 52. (FIGS. 4, 5A and 5B). The tapered portion 46 may also include a threaded region to assist in insertion of the probe 14. The threaded region can also be utilized for collection of cytology samples. All or a portion of the probe 14 may also be coated with a friction reducing coating, e.g., polyfluorocarbon coating, to further aid in the dilation process.

Probe carriage 16 can also be utilized to carry an exfoliation device to sweep the surface of the nipple orifice 44 so as to break up any keratin that may be present, as discussed above. For example, the domed portion 43 of knob 40 (FIG. 3) can be mildly abrasive or an optional brush with bristles 45 can be provided on domed portion 43. The practitioner can use the abrasive surface or the bristles to sweep the nipple surface in preparation for cannulation of the nipple orifice. Incorporation of such an abrasive exfoliation device with the dilator assembly enables the practitioner to conduct the sweeping procedure without need to obtain a different tool, thereby avoiding the possibility that the practitioner may lose sight of the desired nipple orifice to be cannulated while reaching for a different tool or instrument.

Penetration of a mammary duct orifice by the adjustable dilator assembly is described with reference to FIG. 4. A nipple orifice, such as nipple orifice 44 is first located. This may be conducted in any manner, such as through the use of an illuminated nipple cup, magnifier (not shown), or the like. Prior to use, the distal end portion 28 of probe 14 is extended partially beyond the open distal end 24 of sheath 22. Approximately 1/4 to ⅜ of an inch (about 6-10 millimeters) of probe 14 is extended, such that the probe 14, over this relatively long exposed length, is pliable and atraumatic. If the duct is not easily cannulated, such as because sphincter muscle 52 resists the insertion of probe 14 or because of a keratin buildup on the nipple, probe 14 is partially retracted into cannula 21 such that only approximately 1/8 inch (about 3 millimeters) thereof is extended beyond distal end 24. Over such a short length the distal end portion 28 of probe 14 is substantially rigid. The longer extended distal end, which as discussed is relatively pliable, is normally sufficient to bypass the nipple orifice and sphincter muscle, however, in those cases where a more rigid probe is required, such as where the sphincter muscle is difficult to bypass, the exposed length is shortened and the distal end portion 28 made substantially rigid. Accordingly the adjustable dilator assembly 10 serves the dual tasks of a rigid dilator and a pliable dilator. The probe 14 also serves as an obturator for cannula portion 21 so that nipple orifice 44 and sphincter muscle 52 can be easily entered and dilated.

Sheath 22 may also include a tapered portion 54 circumscribing the open distal end 24, to also reduce the possibility of harm to the patient when the cannula 21 is guided into the patient. Similar to probe 14, the sheath 22 can also include a friction reducing coating, e.g., polyfluorocarbon coating.

The nipple orifice 44 and sphincter muscle 52 are penetrated by urging the distal end portion of probe 14, and then sheath 22 into mammary duct 50 past the nipple orifice 44 and sphincter muscle 52 as shown in FIG. 5A. The probe 14 is then further extended into mammary duct 50 as shown in FIG. 5B by further turning probe carriage 16 relative to handle portion 18, as discussed above. The pliable probe 14 thus further penetrates the mammary duct 50 to guide further advancement of sheath 22 into duct 50. As pliable probe 14 is extended further beyond distal end 24 of cannula 21, pliable probe 14 is relatively easily deflected as it is advanced in the mammary duct so as to prevent piercing the mammary duct wall. In this embodiment, the rotation of the probe 14 as it is advanced also serves to prevent the piercing of the wall of mammary duct 50.

After the cannula 21 is positioned as desired, probe carriage 16 and probe 14 are disengaged and removed from dilator assembly 10 and thus cannula 21, and an injection device, such as syringe 60 is attached to dilator assembly 10 as shown in FIG. 6. Syringe 60 is removably secured to handle portion 18 of hollow housing 12 via frictional engagement between female luer taper 19 of handle portion 18, and male luer taper 62 of syringe 60. Fluid already existing within a mammary duct may then be extracted by suction using syringe 60. Alternatively, a rinsing solution, such as saline, with or without a topical anaesthetic such as lidocaine, can be introduced into the mammary duct by a ductal lavage procedure. Because sheath 22 does not include an additional outlet, it also prevents the rinsing solution from prematurely flowing out of the mammary duct. The rinsing solution and any cytological material suspended therein is removed and collected from the duct for analysis by syringe 60. Alternatively, the dilator assembly 10 can be removed and mammary duct fluid collected thereafter through other methods such as a nipple aspiration cup (not shown).

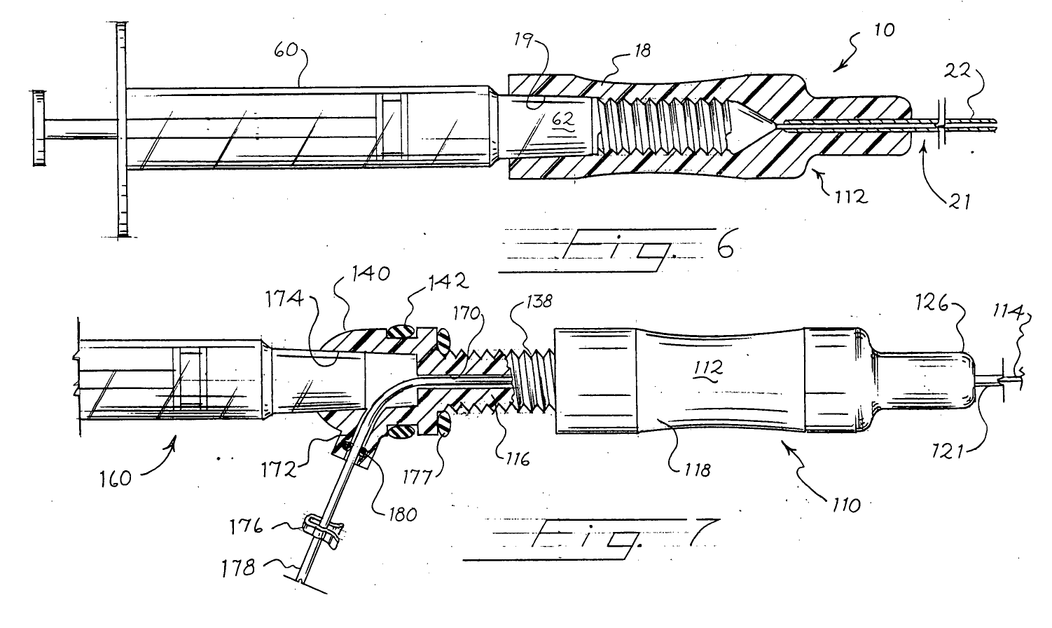

An alternate embodiment of an adjustable dilator assembly is shown in FIG. 7. Similar to the previous embodiment, the adjustable dilator assembly 110 comprises a hollow housing 112, a probe 114 and a probe carriage 116. The housing 112 is substantially the same as hollow housing 12 discussed with respect to FIGS. 1-6. In particular, housing 112 includes a handle portion 118 having a through passage and a cannula 121 extending distally from the handle portion 118. The cannula 121 terminates at an open distal end. The proximal end portion of cannula 121 is secured within a through passage in housing 112 about the distal end portion 126 of the handle portion 118. An interior threaded portion (not shown) is provided in handle portion 118 for threadably receiving threads 138 on probe carriage 116. Unlike the previous embodiment, however, handle portion 118 does not include a socket defining a female luer taper.

Probe carriage 116 includes a turning knob 140 with grip 142 and defines a probe passage 170 and a branch passage 172. When the probe carriage 116 and housing 112 are engaged with one another, probe passage 170 communicates with the through passage in handle portion 118. Probe passage 170 further defines a female luer taper 174 suitable for removably receiving a male luer taper of syringe 160. The inside diameters of branch passage 172 and probe passage 170 are such that probe 114 can be readily inserted into and removed therefrom. Probe 114 further includes a stop such as clip 176, or the like, on the exposed end 178 of probe 114 to limit the extent to which probe 114 may be extended. Stop 176 is shown schematically as being a member securable to the probe, such as clip or star washer that will limit the extent to which probe 114 may be extended through branch passage 172 and probe passage 170. Alternatively, the stop can be an enlarged diameter region of probe 114 or a knotted portion of the probe. An o-ring seal 177 may also be provided to form a seal between housing 112 and probe carriage 116.

Probe 114 is guided into branch passage 172 and probe passage 170 until stop 176 restricts further insertion. The combination of probe 114 and probe carriage 116 is then guided into through passage 120 and sheath 122 of cannula 121, as described with respect to the embodiment illustrated in FIG. 6. As the probe carriage 116 is advanced into housing 112, probe 114 is extended past the distal end of cannula 121. Also, as the probe carriage 116 is rotated relative to the housing 112, the probe 114 carried therewith also rotates, and further facilitates penetration of the mammary duct. If desired, the distal end portion 128 of probe 114 can be provided with a self-threading tip to enhance the penetration and dilation of the mammary duct.

Unlike the embodiment shown in FIG. 6, there is no need to remove the probe carriage 116 from the housing 112 after insertion of the assembly 110. Instead, probe 114 may be withdrawn from dilator assembly 110 by pulling on the exposed end 178 of the probe 114.

Syringe 160 can be used to either introduce fluid to or withdraw fluid from the mammary duct in this manner. In order to prevent or minimize the loss of fluid via branch passage 172 the distal end of probe 114 can be left in branch passage 172. Alternatively, the branch passage 172 may be provided with a peripheral seal, such as a passive valve 180 made from a rubber ring or gasket that is normally closed, i.e., when not accessed, and which closes automatically after access thereof so as to maintain a substantially sealed environment. Passive valve 180 may serve the additional benefit of recovery, by scraping or wiping, of cytological material off the probe 114 that may exist as the probe 114 is withdrawn from the probe carriage 116. As an alternative to the use of an integral syringe, a bulb or balloon-like component 182 can be used as the fluid injection device as shown in FIG. 8. This may be desired because an integral syringe may reduce the fingertip control of the operator because of the added mass of some syringes. In this particular embodiment bulb 182 is unitary with probe support 116, the female luer taper 174 is omitted, and probe passage 170 is in fluid flow communication with bulb 182.

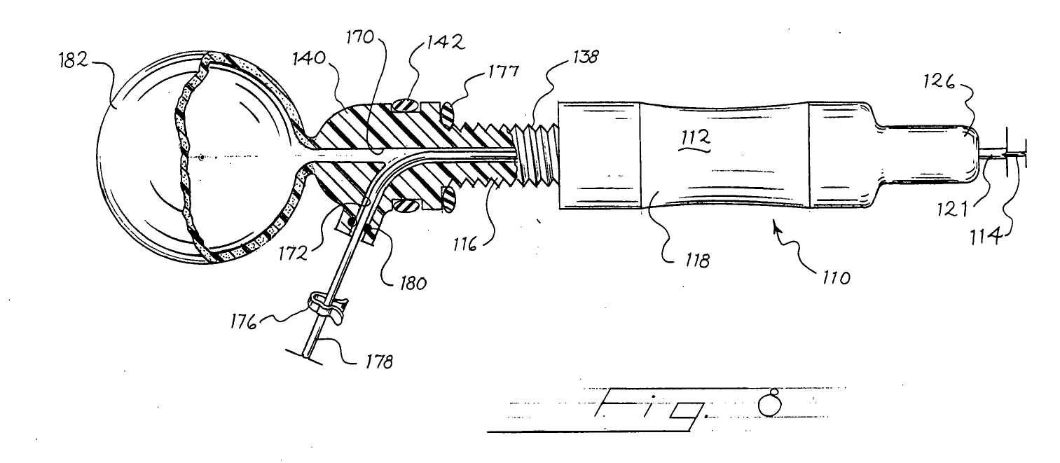

An additional optional feature that may be provided in all embodiments of the adjustable dilator assembly is shown in FIG. 9. The distal end portion 228 of probe 214 is laterally offset. This can be accomplished by heat bending the probe 214 such that when the distal end portion 228 is in a relaxed state, it will include the lateral offset. Because of the pliability of the distal end portion 228 of probe 214, the probe 214 is straightened when in the sheath 222, such as shown in FIG. 9A. The offset distal end portion 228 of probe 214 can serve the further purpose of acting as a brush by rotating the assembly to loosen and dislodge cellular material from the intraductal epithelium of the mammary duct, and thereby increase the yield of cells and cellular debris.

Yet another embodiment of the present invention is shown in FIG. 10. Adjustable dilator assembly 210 includes a hollow housing 212 which terminates into a cannula 221, and a probe 214 mounted for axial movement within the housing 212 and through the cannula 221. The proximal end of the probe 214 is secured to a probe carriage, such as plunger 262 of syringe 260. Syringe 260 is also removably secured to hollow housing 212. As plunger 262 is moved distally towards hollow housing 212, probe 214 is further extended through the hollow housing 212 and cannula 221. As plunger 262 is moved proximally away from hollow housing 212, probe 214 is fully retracted into hollow housing 212, thereby clearing cannula 221 for withdrawal of liquid from the mammary duct. Hollow housing 212 and syringe barrel 268 can also serve as a container for carrying the fluid sample as well as the probe 214, which may include cytological material adhered thereto, especially if as discussed above, the probe was used as a brush. If desired, a portion of probe 214, especially the distal end thereof, can be porous to further facilitate retention of cytological material.

Preferably, syringe 260 includes a syringe stop 264 which engages an annular ridge 266 positioned about the proximal end of syringe barrel 268. Syringe stop 264 prevents the plunger 262 from exiting the syringe barrel 268. Syringe 260 further includes a syringe detent 270 on plunger 262 which is engaged as syringe detent 270 exits the proximal end of syringe barrel 268. The cooperation of syringe detent 270 and syringe stop 264 enable a practitioner to withdraw plunger 262 from barrel 268 to a position that is fixable, so as to prevent the accidental re-injection of the fluid withdrawn from the mammary duct or the inadvertent loss or contamination of the sample through removal of the plunger 262 from the barrel 268. Detent 270 is disengageable to permit collected fluid to be ejected from syringe 270 under desired conditions. Syringes having a syringe stop and a syringe detent such as described herein are commercially available under the trade name VACLOK™ from Qosina Corporation, Edgewood, N.Y.

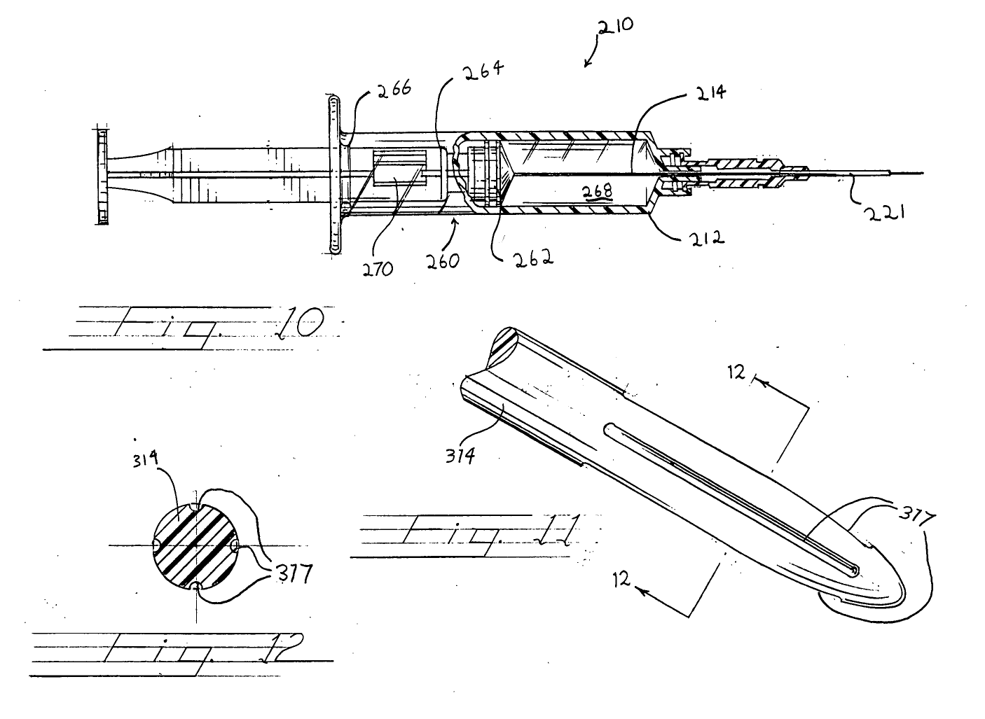

An additional optional feature that may be included in any of the embodiments previously discussed is shown in FIGS. 11 and 12. As discussed with respect to FIGS. 9 and 9A, the distal end of the probe may act as a brush for loosening or dislodging cytological material from the intraductal epithelium of the mammary duct. After such a brushing biopsy, cytological material may also be found on the distal end of the probe. As such, the distal end portion of probe 314 is provided with a brushing device that includes grooved regions 317 at spaced intervals around the periphery thereof. Such grooved regions 317 provide a roughened surface to enhance the brushing effect of the probe 314 as well as to provide a collection recess for harvesting cytological material. Along with fluid obtained from a ductal lavage procedure, any cytological material harvested on probe 314, and more likely in grooved regions 317, is also analyzed.

The foregoing description is to be taken as illustrative, but not limiting. Still other variants within the spirit and scope of the present invention will readily present themselves to those skilled in the art.

I claim:

1. An adjustable dilator assembly comprising:
an elongated hollow housing that terminates at one end in a cannula and defines a longitudinal axial through passage; and
a probe mounted for adjustable axial movement within the housing and cannula.

2. The adjustable dilator assembly of claim 1, wherein at least a distal end portion of the probe is pliable.

3. The adjustable dilator assembly of claim 2, wherein the distal end portion of the probe is offset laterally.

4. The adjustable dilator assembly of claim 1, wherein the probe is fixedly secured to a probe carriage axially adjustable with the hollow housing.

5. The adjustable dilator assembly of claim 4, wherein the probe is integral with the probe carriage.

6. The adjustable dilator assembly of claim 4, wherein the probe and the probe carriage are unitary.

7. The adjustable dilator assembly of claim 4, wherein the hollow housing is provided with internal threads and the probe carriage is provided with external threads for threadable engagement therewith.

8. The adjustable dilator assembly of claim 4, wherein the probe carriage is elongated and has a distal end portion as well as a proximal end portion, wherein the probe is mounted to the distal end portion of the probe carriage, and wherein the proximal end portion carries an exfoliation device.

9. The adjustable dilator assembly of claim 8, wherein the exfoliation device is an abrasive surface on said distal end portion.

10. The adjustable dilator assembly of claim 8, wherein the exfoliation device is a brush carried by said distal end portion.

11. The adjustable dilator assembly of claim 1, wherein a socket for receiving an injection device is present at a proximal end portion of the hollow housing.

12. The adjustable dilator assembly of claim 11, wherein the socket defines a female luer taper.

13. The adjustable dilator assembly of claim 1, wherein a distal end portion of the probe includes a taper and terminates in a rounded tip.

14. The adjustable dilator assembly of claim 1, wherein the probe is removably secured to a probe carriage.

15. The adjustable dilator assembly of claim 11, wherein the probe is retractable from the probe carriage.

16. The adjustable dilator assembly of claim 1, wherein the probe is carried by a probe carriage that defines a through passage in fluid communication with the through passage of the hollow housing.

17. The adjustable dilator assembly of claim 16, wherein the probe carriage is provided with a socket for receiving an injection device.

18. The adjustable dilator assembly of claim 17, wherein the socket defines a female luer taper.

19. The adjustable dilator assembly of claim 16, wherein the probe carriage further includes an injection device unitary therewith.

20. The adjustable dilator assembly of claim 16, wherein the probe carriage further includes a branch passage.

21. The adjustable dilator assembly of claim 20, wherein the probe includes a stop.

22. The adjustable dilator assembly of claim 20, wherein the branch passage includes a peripheral seal.

23. The adjustable dilator assembly of claim 1, wherein a distal end portion of the probe includes a brushing device.

24. The adjustable dilator assembly of claim 1, wherein a distal end portion of the probe is porous.

25. An adjustable dilator assembly comprising:
an elongated hollow housing that terminates at one end in a cannula, has an opposite open end, and defines an internally threaded axial through passageway in communication with the cannula;
an elongated, externally threaded probe carriage threadably received into the elongated hollow housing through the open end thereof, having a threaded distal end portion and a proximal end portion provided with a grip; and
a probe mounted to the probe carriage at the distal end portion thereof, extendable through the cannula, and mounted for adjustable axial movement within the housing and the cannula in response to manipulation of the probe carriage.

26. The adjustable dilator assembly in accordance with claim 22 wherein the proximal end portion carries an exfoliation device.

27. The adjustable dilator assembly in accordance with claim 26, wherein the exfoliation device is a brush.

28. The adjustable dilator assembly in accordance with claim 26, wherein the exfoliation device is an abrasive surface.
